(19) United States
(12) Patent Application Publication
(10) Pub. No.: US 2017/0113014 A1
(43) Pub. Date: Apr. 27, 2017

(54) RESPIRATORY ASSISTANCE APPARATUS

(71) Applicant: METRAN CO., LTD., KAWAGUCHI-SHI, SAITAMA (JP)

(72) Inventor: Kazufuku NITTA, Kawaguchi-shi, Saitama (JP)

(21) Appl. No.: 15/129,173

(22) PCT Filed: Mar. 26, 2015
(86) PCT No.: PCT/JP2015/059469

§ 371 Date: Sep. 26, 2016

(30) Foreign Application Priority Data
Mar. 26, 2014 (JP) 2014-063853

(51) Int. Cl. A61M 16/20; A61M 16/00; A61M 16/16; A61M 16/06

(52) U.S. Cl. CPC A61M 16/202; A61M 16/0666; A61M 16/208; A61M 16/0003; A61M 16/0066; A61M 16/16; A61M 2016/0027; A61M 2209/088; A61M 2016/0033

(57) ABSTRACT

A respiratory assistance apparatus guides gas from a gas supply source to a connection part via an inspiratory pathway. A check valve that allows the gas from the gas supply source to pass to the connection part side and prevents exhaled air discharged from the nose or mouth from flowing into the gas supply source side is arranged on the inspiratory pathway. An expiratory valve is arranged on the inspiratory pathway closer to the connection part than the check valve is, and the expiratory valve is opened to discharge the exhaled air from the inspiratory pathway during expiration. This eases a respiratory load on the patient and reduces contamination of the apparatus.

### TECHNICAL FIELD

The present invention relates to a respiratory assistance apparatus.

### BACKGROUND ART

Sleep apnea syndrome (SAS) is caused by airway muscles relaxing during sleep to lower the tongue root and/or the soft palates and block the airway. Patients of this type use a respiratory assistance apparatus that includes a blower for applying a positive pressure to the airway. For example, see Metran Co., Ltd., [online], Products>Jusmine, [Searched on 27 Jan. 2014], the Internet (http://www.metran.co.jp/products/products2/190.html). The respiratory assistance apparatus sends out compressed air supplied from the blower into the patient's airway as inspiratory air. To suppress drying of the airway here, the compressed air is humidified on the inspiratory pathway before supplied to the patient.

A plurality of air holes are formed in a portion of the inspiratory airway near the patient. Exhaled air from the patient is exhaled against the inflow of the compressed air and discharged through the air holes.

### SUMMARY OF INVENTION

### Technical Problem

Respiratory assistance apparatuses have been miniaturized in recent years. The inspiratory pathway can be shortened accordingly. A publicly unknown research by the present inventors has found that the shorter the inspiratory pathway is, the smaller the capacity of the inspiratory pathway becomes and the more likely the exhaled air is to flow back to the blower through the inspiratory pathway. This results in a high expiratory resistance to the patient, and there has been a problem of being prone to cause a feeling of dislike.

There has been another problem that if the exhaled air of the patient flows back to the blower, the blower can be contaminated by the exhaled air.

In addition, since the air holes for discharging the exhaled air are formed in the inspiratory pathway, the compressed inspiratory air from the blower constantly leaks from the air holes. For example, if the blower supplies 80 liters of compressed air per minute, approximately 30 liters of it is let out from the air holes. Therefore, there has been a problem that the power of the blower is wasted.

The present invention has been achieved in view of the foregoing problems, and an object of the present invention is to provide a respiratory assistance apparatus that can reduce contamination of a gas supply source and reduce an expiratory load on the patient even if the inspiratory pathway from the gas supply source is short.

### Solution to Problem

To achieve the foregoing object, a respiratory assistance apparatus includes: a gas supply source configured to supply a gas; a connection part that is connected to a nose or mouth and configured to supply the gas thereto; an inspiratory pathway configured to make the gas supply source communicate with the connection part and guide the gas; a backflow prevention mechanism that is arranged on the inspiratory path and configured to allow the gas of the gas supply source to pass to the connection part side and to prevent exhaled air discharged from the nose or mouth via the connection part from flowing into a side of the gas supply source; and an air hole that is formed in a pathway constituting member constituting the inspiratory pathway and configured to discharge the exhaled air, the air hole being formed closer to the connection part than the backflow prevention mechanism is.

In the foregoing respiratory assistance apparatus, the backflow prevention mechanism is a check valve that operates mechanically by using a pressure or flow of the exhaled air.

In the foregoing respiratory assistance apparatus, the backflow prevention mechanism is an actuated valve that operates by using an electrical signal obtained by detecting the exhaled air.

The foregoing respiratory assistance apparatus further includes an expiratory valve configured to open and close the air hole, wherein the expiratory valve closes the air hole during inspiration and opens the air hole during expiration.

The foregoing respiratory assistance apparatus includes an expiratory sensor configured to detect the exhaled air, wherein the expiratory valve opens and closes according to expiration detection of the expiratory sensor.

In the foregoing respiratory assistance apparatus, the expiratory sensor is arranged on the pathway constituting member, closer to the connection part than the backflow prevention mechanism is.

In the foregoing respiratory assistance apparatus, the expiratory sensor is an air pressure meter.

In the foregoing respiratory assistance apparatus, an exhaust hole configured to release the gas of the gas supply source is formed in the inspiratory pathway between the gas supply source and the backflow prevention mechanism.

The foregoing respiratory assistance apparatus further includes an exhaust valve configured to open and close the exhaust hole, wherein the exhaust valve closes the exhaust hole during inspiration and opens the exhaust hole during expiration.

In the foregoing respiratory assistance apparatus, the inspiratory path between the gas supply source and the connection part has a length of 500 mm or less.

In the foregoing respiratory assistance apparatus, the inspiratory path between the check valve and the connection part has a length of 300 mm or less.

In the foregoing respiratory assistance apparatus, the gas supply source is a blower, and the blower is fixed to a human body.

In the foregoing respiratory assistance apparatus, the blower is fixed to a head.

The foregoing respiratory assistance apparatus includes a humidifying device that is configured to humidify the gas and is arranged on the pathway constituting member, closer to the gas supply source than the backflow prevention mechanism is.

### Advantageous Effects of Invention

According to the present invention, an excellent effect of easing a respiratory load on a patient and reducing contamination of the apparatus can be obtained.

### BRIEF DESCRIPTION OF DRAWINGS

FIG. 1 is a sectional view of a respiratory assistance apparatus according to an embodiment of the present invention.

FIG. 2 is a side view of the respiratory assistance apparatus.

FIG. 3 is a block diagram showing a hardware configuration of a control unit.

FIG. 4 is a schematic diagram showing a functional configuration of the control unit.

FIGS. 5(A) and 5(B) are sectional views of a chamber portion, FIG. 5(A) showing a state when an expiratory valve is opened, FIG. 5(B) showing a state when the expiratory valve is closed.

FIG. 6 is a use state diagram of the respiratory assistance apparatus.

FIG. 7 is a use state diagram of the respiratory assistance apparatus for describing moment.

FIG. 8 is a use state diagram showing a respiratory assistance apparatus according to another example of the present embodiment.

FIG. 9 is a use state diagram showing a respiratory assistance apparatus according to another example of the present embodiment.

FIGS. 10(A) and 10(B) are sectional views of the chamber portion of a respiratory assistance apparatus according to another example of the present embodiment, FIG. 10(A) showing a state when the expiratory valve is opened, FIG. 10(B) showing a state when the expiratory valve is closed.

### DESCRIPTION OF EMBODIMENTS

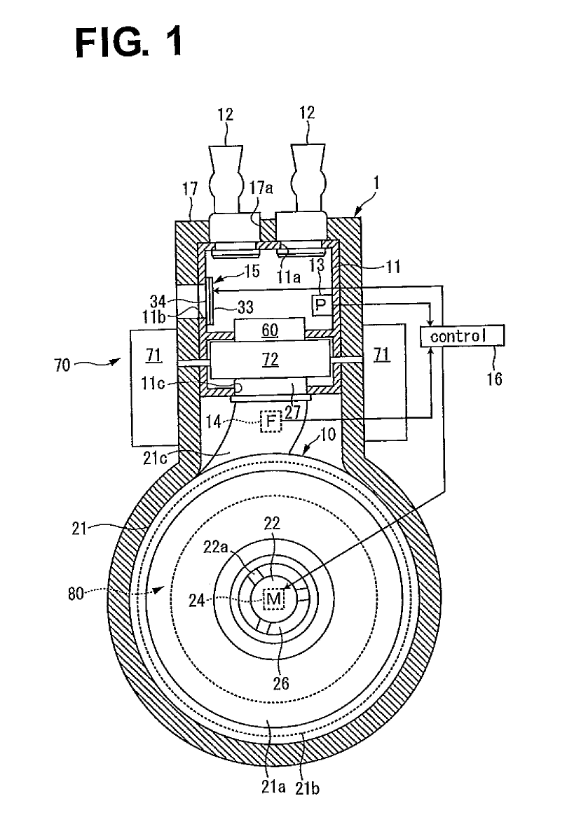

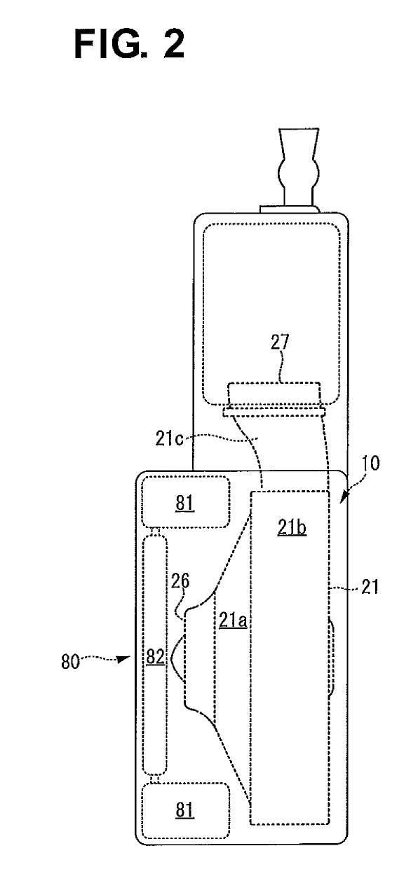

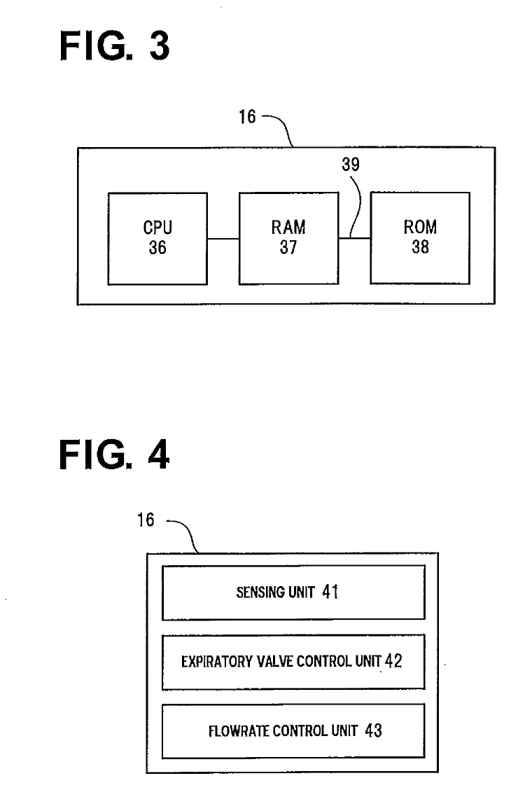

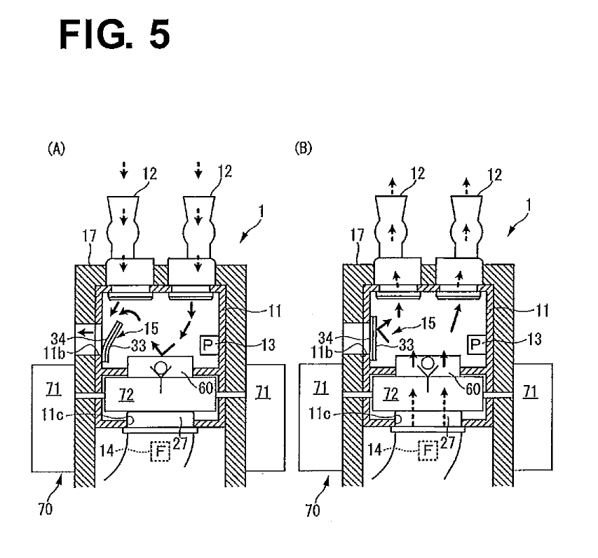

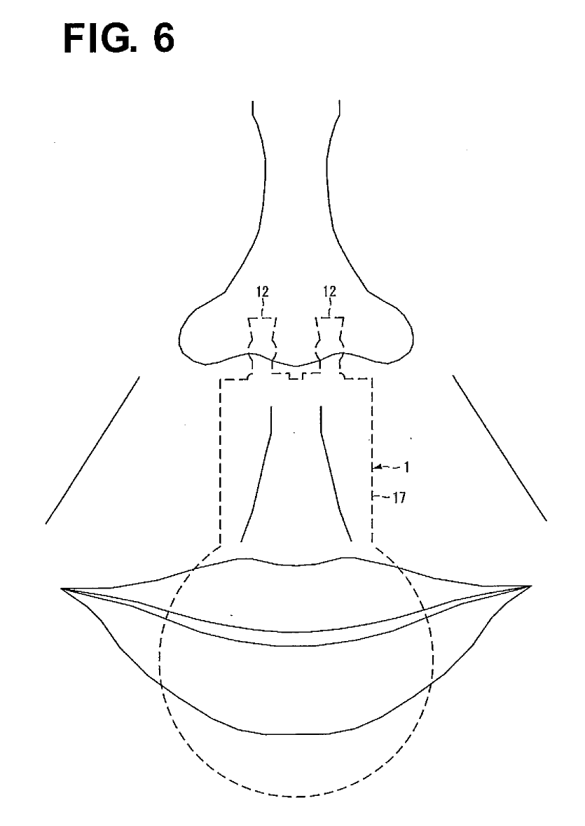

A respiratory assistance apparatus 1 according to an embodiment of the present invention will be described in detail below with reference to the drawings. FIG. 1 is a sectional view of the respiratory assistance apparatus 1, and FIG. 2 is a side view thereof. FIG. 3 is a block diagram showing a hardware configuration of a control unit 16. FIG. 4 is a schematic diagram showing a functional configuration of the control unit 16. FIGS. 5(A) and 5(B) are sectional views of a chamber 11 portion constituting an inspiratory pathway. FIG. 5(A) shows a state when an expiratory valve 15 is opened, and FIG. 5(B) shows a state when the expiratory valve 15 is closed. FIG. 6 is a use state diagram of the respiratory assistance apparatus 1. In the drawings, some components, hatching representing cross sections, and the like are appropriately omitted for simplification. In the drawings, members are expressed in appropriately exaggerated sizes.

The respiratory assistance apparatus 1 shown in FIG. 1 is intended to produce a positive pressure in an airway, and is used by a patient with a respiratory disorder. This respiratory assistance apparatus 1 is of so-called prong type. Specifically, the respiratory assistance apparatus 1 includes a blower 10 which serves as a gas supply source, a chamber 11 which constitutes a part of an inspiratory pathway, a pair of prongs 12 which serves as a connection part with a human body, an air pressure meter 13 which functions as an expiratory sensor for detecting exhaled air, a check valve 60, a downstream humidifier 70, an upstream humidifier 80, a flowmeter 14, an expiratory valve 15, the control unit 16, and a case 17. While an example of the prong type to be connected to the nose of the human body is described here, a mask type structure to be connected to the mouth may be employed.

The blower 10 is connected to the pair of prongs 12 via the chamber 11. This blower 10 sends out air to the nasal cavity (airway) of the user through the pair of prongs 12. The blower 10 thereby produces a positive pressure in the airway. An impeller (omitted in the diagram) and a motor M are built in a housing 21 of the blower 10.

As shown in FIG. 2, the housing 21 is a resin-molded main body of the blower 10, and is constituted by an upper portion 21a having a generally truncated conical external shape, a lower portion 21b having a generally cylindrical external shape, and a discharge pipe 21c extending sideways from the lower portion 21b. The upper portion 21a curves smoothly upward. The upper portion 21a has a circular inspiratory port 26 in its top end. The discharge pipe 21c has a discharge port 27 in its extremity. The blower 10 takes in air from the inspiratory port 26 and sends out the air from the discharge port 27. The air is not restrictive, and other gases such as medicine-mixed air and oxygen may be used.

The upstream humidifier 80 is arranged on the upstream side (inspiratory side) of the inspiratory port 26, i.e., between the case 17 and the inspiratory port 26. The upstream humidifier 80 humidifies the gas taken into the blower 10 on the upstream side of the blower 10 to such a degree that moisture does not condense in the blower 10. Specifically, the upstream humidifier 80 includes a container 81 which contains water for humidification, and a water permeable member 82 which is arranged upstream of the blower 10 and evaporates water supplied from the container 81. The upstream humidifier 80 may be of integrated type which is fixed to the case 17. The upstream humidifier 80 may have a structure such that the container 81 containing the water for humidification is provided separate from the case 17 and the water is supplied to the water permeable member 82 through piping. For example, see the humidifier of Japanese Patent No. 4771711 for details of the upstream humidifier 80.

Returning to FIG. 1, the chamber 11 serves as the pathway of the air (inspiratory air) sent out from the blower 10. The chamber 11 has a pair of air holes 11a to which the prongs 12 are attached, an air hole 11b which is opened and closed by the expiratory valve 15, and a connection port 11c to which the discharge port 27 of the blower 10 is connected. The pair of prongs 12 are nozzles to be inserted into the nose of the user. The pair of prongs 12 are detachably attached to the air holes 11a of the chamber 11. The pair of prongs 12 thereby guides the air sent out from the blower 10 to the nasal cavity of the user as inspiratory air. The pair of prongs 12 also guides exhaled air of the user to the chamber 11. The distance of the inspiratory pathway constituted by the chamber 11, i.e., the distance from the discharge port 27 of the blower 10 to the prongs 12 is preferably within 500 mm, desirably within 310 mm. Here, the distance is within 50 mm, or approximately 30 mm.

The downstream humidifier 70 is connected to the discharge port 27 of the blower 10, and humidifies the gas sent out from the discharge port 27 on the downstream side of the blower 10 to such a degree that the airway of the user is prevented from drying (such a degree that moisture condenses). Specifically, the downstream humidifier 70 includes containers 71 which are fixed to and arranged on the sides of the case 17 and contain water for humidification, and a water permeable member 72 which is arranged downstream of the discharge port 27 in the chamber 11 and evaporates water supplied from the containers 71. The downstream humidifier 70 may be of integrated type which is fixed to the case 17. The downstream humidifier 70 may be of separate type in which a container 71 containing water for humidification is installed in a place remote from the case 17 and the water is supplied through piping. The water containers 81 and 71 of the upstream humidifier 80 and the downstream humidifier 70 may be made common. For example, see the humidifier of Japanese Patent No. 4771711 for details of the downstream humidifier 70.

The check valve 60 constitutes a backflow prevention mechanism in the present invention. The check valve 60 is arranged downstream of the downstream humidifier 70 in the chamber 11, and guides the gas passed through the downstream humidifier 70 to the prong 12 side. On the other hand, if exhaled air discharged from the prongs 12 into the chamber 11 attempts to flow into the check valve 60 side, the check valve 60 blocks the flow. Specifically, suppose that the flow of the gas from the blower 10 to the chamber 11 is a forward direction. The check valve 60 has a passive structure of mechanically blocking the flow if the flowing direction of the gas is reversed. In other words, the check valve 60 physically uses the pressure of the exhaled air (pressure difference between the exhaled air and the supplied gas) or the flow of the exhaled air to operate mechanically. The distance of the inspiratory pathway from the check valve 60 to the prongs 12 is preferably 300 mm or less, desirably 100 mm or less, more desirably 50 mm or less. Here, the distance is approximately 20 mm. While an example of the check valve that operates mechanically with the exhaled air is described here, a sensor for detecting the exhaled air, such as the air pressure meter 13, may be used to electrically operate an actuated valve with its electrical signal. For example, a solenoid valve may be used as the actuated valve. A piezo element like that of the expiratory valve to be described later may be used.

The air pressure meter 13 is arranged downstream of the check valve 60 in the chamber 11. The air pressure meter 13 measures the air pressure in the chamber 11 and outputs the measurement result to the control unit 16 in the form of a signal. The flowmeter 14 is arranged upstream of the check valve 60, or more specifically, in the discharge pipe 21c of the blower 10. The flowmeter 14 measures the flowrate of the air sent out from the blower 10 and outputs the measurement result to the control unit 16 in the form of a signal.

The expiratory valve 15 is arranged inside the chamber 11 to block the air hole 11b formed in the chamber 11. The expiratory valve 15 opens the air hole 11b at predetermined timing to release the exhaled air guided into the chamber 11 to the atmosphere. The expiratory valve 15 otherwise closes the air hole 11b to prevent the air (inspiratory air) from the blower 10 from flowing out.

The expiratory valve 15 has a monomorph (unimorph) structure formed by stacking a piezo element (piezoelectric element) 33, which makes a displacement according to the amount of voltage applied thereto, on a metal plate 34. The expiratory valve 15 is a valve having a cantilever structure. The expiratory valve 15 thus opens and closes as the piezo element 33 makes a displacement to curve or stretch out. More specifically, the piezo element 33 of the expiratory valve 15 makes a displacement to be separated from or approach into contact with the inner surface of the chamber 11, whereby the piezo element 33 opens and closes the air hole 11b formed in the chamber 11 by itself.

Specifically, as shown in FIG. 5(A), when in an initial state where no voltage is applied to the piezo element 33, the expiratory valve 15 takes the shape of curving toward the inside of the expiratory pathway to open the air hole 11b formed in the chamber 11. As shown in FIG. 5(B), when a voltage is applied to the piezo element 33, the expiratory valve 15 takes the shape of stretching out to close the air hole 11b formed in the chamber 11. The expiratory valve 15 is appropriately fixed, for example, by a screw (not shown).

While the expiratory valve 15 of monomorph structure is discussed here, it will be understood that a bimorph structure including a laminate of two piezo elements may be employed. The stroke by which the expiratory valve 15 makes a displacement is preferably 2 mm or greater and 3 mm or less.

As shown in FIG. 3, the control unit 16 includes a CPU 36, a first storage medium 37, a second storage medium 38, and a bus 39.

The CPU 36 is a so-called central processing unit. Various programs are executed to implement various functions of the present control unit 16. The first storage medium 37 is a so-called RAM (random access memory) and used as a work area of the CPU 36. The second storage medium 38 is a so-called ROM (read only memory) and stores the programs to be executed by the CPU 36. The bus 39 serves as wiring for integrally connecting the CPU 36, the first storage medium 37, the second storage medium 38, and the like for communication.

As shown in FIG. 4, the control unit 16 includes a sensing unit 41, an expiratory valve control unit 42, and a flowrate control unit 43 as its functional configuration. The sensing unit 41 constantly obtains and transmits sensing data of the air pressure meter 13 to the expiratory valve control unit 42. The sensing unit 41 also constantly obtains and transmits sensing data of the air pressure meter 13 and the flowmeter 14 to the flowrate control unit 43. The expiratory valve control unit 42 refers to the sensing data of the sensing unit 41, and controls a control signal to the expiratory valve 15 to approach a target opening amount. The flowrate control unit 43 refers to the sensing data of the sensing unit 41, and controls a control signal to the motor of the blower 10 to approach a target flowrate value.

In FIG. 1, the control unit 16 is shown outside the case 17 for ease of understanding. In fact, the control unit 16 is accommodated in the case 17.

Next, a control example of the expiratory valve 15 in the respiratory assistance apparatus 1 will be described with reference to FIGS. 5 and 6.

If the user exhales, the pressure in the chamber 11 increases. Here, since the backflow of the exhaled air to the blower 10 side is blocked by the function of the check valve 60, the pressure of the chamber 11 increases quickly. In particular, the small capacity of the chamber 11 (inspiratory pathway) between the check valve 60 and the prongs 12 is effectively used to make the pressure in the chamber 11 increase sharply by blocking the exhaled air attempting to flow back to the blower 10 side with the check valve 60. If the pressure in the chamber 11 increases, the increased pressure value is quickly sensed by the air pressure meter 13. The sensing data is output to the control unit 16. The control unit 16 controls the expiratory valve 15 on the basis of the sensing data. More specifically, the control unit 16 operates the expiratory valve 15 to open the air hole 11b of the chamber 11 (see FIG. 5(A)). The exhaled air is released from the air hole 11b. Here, the motor 24 of the blower 10 may be controlled to reduce the flowrate of or stop the blower 10.

The release of the exhaled air reduces the pressure in the chamber 11. If the pressure in the chamber 11 decreases, the decreased pressure value is sensed by the air pressure meter 13. The sensing data is output to the control unit 16. The control unit 16 controls the expiratory valve 15 on the basis of the sensing data. More specifically, the control unit 16 operates the expiratory valve 15 to close the air hole 11b (see FIG. 5(B)). This forms a closed space inside the chamber 11 to enable an inspiratory operation. If the blower 10 is maintained running, the gas is naturally supplied to the prong 12 side. If the blower 10 is stopped during expiration, the driving of the blower 10 may be started at this timing.

If the user inhales, the pressure in the chamber 11 decreases. If the pressure in the chamber 11 decreases, the decreased pressure value is sensed by the air pressure meter 13. The sensing data is output to the control unit 16. The control unit 16 controls the motor 24 of the blower 10 on the basis of the sensing data. More specifically, the control unit 16 drives the motor 24 to increase the flowrate of the blower 10. The blower 10 may be turned on at the timing of detection of this inspiratory operation.

The blower 10 sends out air as inspiratory air, whereby the pressure in the chamber 11 is increased. If the pressure in the chamber 11 increases, the increased pressure value is sensed by the air pressure meter 13. The sensing data is output to the control unit 16. The control unit 16 determines the end timing of the inspiration on the basis of the sensing data, and controls the motor 24 of the blower 10. More specifically, the control unit 16 stops or reduces the speed of the motor 24 to stop or suppress the air sent out from the blower 10 as the inspiratory air. Subsequently, the same expiratory operation and inspiratory operation are repeated.

The case 17 includes the upstream humidifier 80 which is arranged on the inspiratory port 26 of the blower 10. This upstream humidifier 80 is expected to also provide the effect of absorbing noise from the blower 10. A porous member (such as an interconnected cell sponge) for preventing intrusion of dust is preferably arranged further upstream of the upstream humidifier 80.

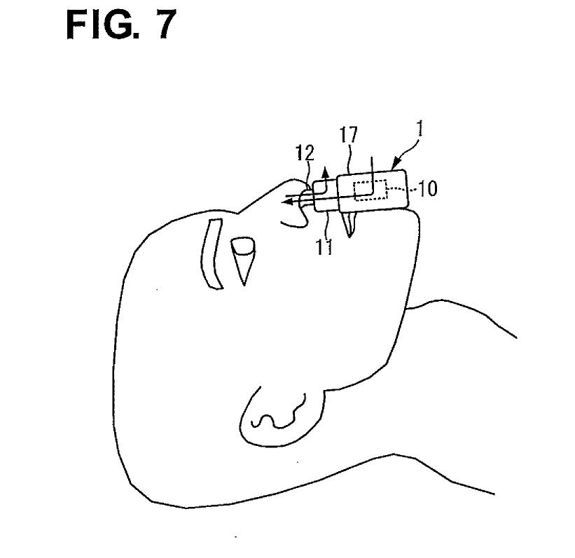

Next, a use state of the respiratory assistance apparatus 1 will be described with reference to FIGS. 6 and 7.

The respiratory assistance apparatus 1 is used with the pair of prongs 12 inserted into the nasal cavity. The portion of the case 17 where the blower 10 is accommodated is placed on the mouth of the user and makes contact with the mouth of the user. That is, the blower 10 is in indirect contact with the mouth of the user. According to such a respiratory assistance apparatus 1, the distance from the center axis of the body of the user to the center of gravity of the blower 10 can be made smaller than heretofore. This can reduce the moment of the blower 10 if the user in a recumbent position rolls over or turns the face. Since the blower 10 is placed on the mouth, the blower 10 will not be pressed against the pillow with the face if the user rolls over or turns the face. As a result, burdens on the user can be reduced.

The blower 10 (the portion of the case 17 where the blower 10 is accommodated) holds the user's mouth, whereby the user can be assisted in keeping the mouth closed. This results in a mouth-closed state which is desirable during nasal respiration, and burdens on the user can be reduced. The contact with the mouth may be direct or indirect.

According to the present respiratory assistance apparatus 1, the expiratory valve 15 can be closed to make the interior of the pathway airtight during inspiration. This can reduce the leakage of the gas supplied from the blower 10 from the expiratory valve 15 during inspiration.

According to this respiratory assistance apparatus 1, the expiratory valve 15 includes the piezo element 33, and the opening amount thereof can be finely adjusted. This can prevent a sudden change in the flowrate of the exhaled air released from the expiratory valve 15. The inclusion of the piezo element 33 in the expiratory valve 15 provides high responsiveness. Specifically, if a solenoid valve is used as the expiratory valve 15, the expiratory valve 15 opens and closes in a time of approximately 8 msec to 10 msec. If the expiratory valve 15 includes the piezo element 33 as in the foregoing embodiment, the expiratory valve 15 can be opened and closed in a time as short as 100 μsec or so. During expiration, the check valve 60 can be used to make the pressure in the chamber 11 increase sharply to increase the responsiveness of the air pressure meter 13. The expiratory valve 15 can be opened almost simultaneously with the response of the air pressure meter 13. This can ease a load on the user during expiration.

Since the expiratory valve 15 includes the piezo element 33, the expiratory valve 15 has a longer endurance time and is more durable than when a solenoid valve is employed as the expiratory valve 15. The inclusion of the piezo element 33 in the expiratory valve 15 also enables miniaturization and weight reduction of the respiratory assistance apparatus 1 as compared to such cases as where a solenoid valve is employed as the expiratory valve 15. The gravity of the respiratory assistance apparatus 1 on the face of the user and the like can thus be reduced to reduce burdens on the user.

According to this respiratory assistance apparatus 1, the check valve 60 prevents the exhaled air from flowing back toward the downstream humidifier 70 and the blower 10. The contamination of the apparatus by the exhaled air can thus be suppressed. As a result, the maintenance frequency of the respiratory assistance apparatus 1 can be reduced.

According to the present respiratory assistance apparatus 1, the upstream humidifier 80 performs humidification in advance before the humidification by the downstream humidifier 70. This can increase the amount of humidification of the gas supplied to the airway of the user. The inspiratory air can thus be sufficiently humidified even if the distance of the inspiratory pathway from the blower 10 to the prongs 12 is short and the downstream humidifier 70 is not capable of sufficient humidification.

Suppose that there is provided no downstream humidifier 70 and only the upstream humidifier 80 is used to perform humidification to provide the amount of humidification for preventing the drying of the user's airway. In such a case, moisture will condense inside the blower 10. According to the present respiratory assistance apparatus 1, the upstream humidifier 80 desirably provides the amount of humidification to such a degree that moisture does not condense in the blower 10, before the downstream humidifier 70 achieves the amount of humidification for preventing the drying of the user's airway (the amount of humidification for causing condensation). No condensation therefore occurs in the blower 10.

Here, the motor built in the blower 10 functions as a heater, which can also prevent the occurrence of condensation in the blower 10. The amount of humidification by the upstream humidifier 80 therefore can be increased. Consequently, the amount of humidification for preventing the drying of the user's airway can be achieved even if the inspiratory pathway from the blower 10 is short and the amount of humidification by the downstream humidifier 70 is small. It will be understood that a heater may be built in the blower 10 aside from the motor.

This respiratory assistance apparatus 1 can be used as a home artificial respirator by a patient with sleep apnea syndrome or the like. The respiratory assistance apparatus 1 can also be used as an artificial respirator in medical institutions. The blower serving as the gas supply source may be replaced with an oxygen cylinder or the like.

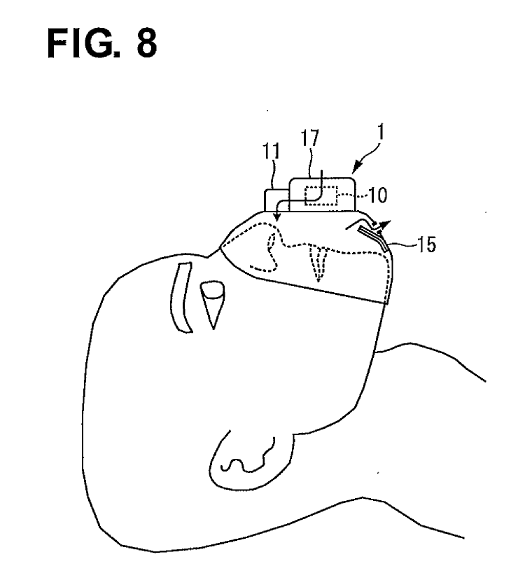

In the present embodiment described above, the prongs 12 are used as the connection part with the patient, and an example of the case of supplying the gas to the nose of the patient by using the same has been described. However, as shown in FIG. 8, a mask covering both the mouth and the nose may be used as the connection part. In such a case, the blower 10 is arranged outside or inside the mask, and the check valve is arranged on the way of the inspiratory pathway (chamber 11) from the blower to the internal space of the mask. The gas is supplied to the mask via the chamber 11. The expiratory valve 15 may be arranged on a wall of the mask.

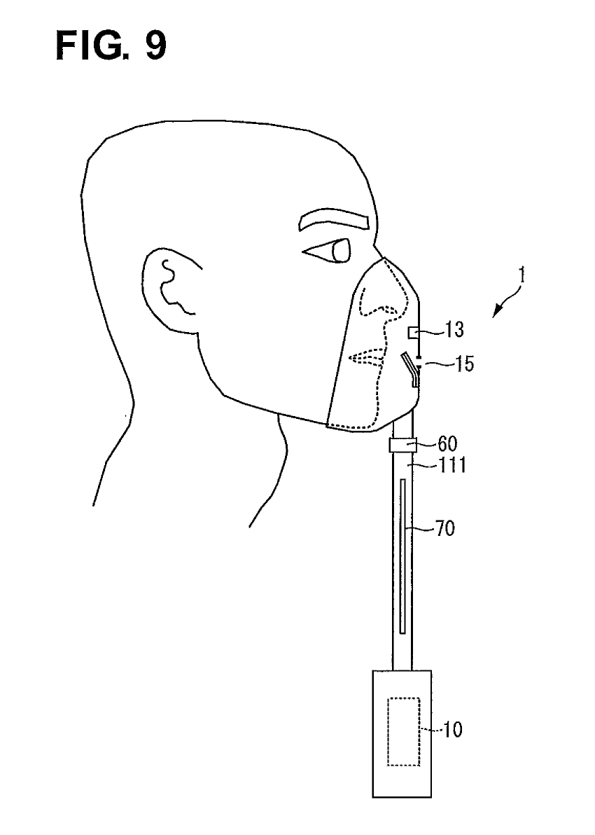

In the present embodiment, the blower 10 and the chamber 11 constituting the inspiratory pathway are described to be integrated with each other. For example, like the respiratory assistance apparatus 1 shown in FIG. 9, a pipe 111 of bellows structure may be employed as the pathway constituting member for constituting the inspiratory pathway, and the blower 10 and a mask (or prongs) may thereby be connected. In such a configuration, the blower 10 can be fixed to a place other than the mouth on the head of the patient, or the chest or an arm, or may be arranged by the bed. Since the check valve 60 can prevent the exhaled air from flowing back to the blower 10 through the pipe 111, the contamination of the blower 10 can be suppressed.

Here, the check valve 60 is preferably located on the pipe 111 as close to the mask (or prongs) as possible. The distance therebetween is preferably 300 mm or less, desirably 100 mm or less, more desirably 50 mm or less. Arranging the check valve 60 and the mask (or prongs) close to each other can reduce the amount of backflow of exhaled air containing a lot of carbon dioxide to the pipe 111 side, and suppress the patient inhaling his/her own exhaled air again at the time of the next inspiration. As already mentioned, the reduced capacity between the check valve 60 and the mask can also make the pressure increase during expiration quicker, whereby the detection time of the exhaled air by the air pressure meter 13 can be reduced. Consequently, the expiratory valve 15 arranged on the wall of the mask can be quickly opened.

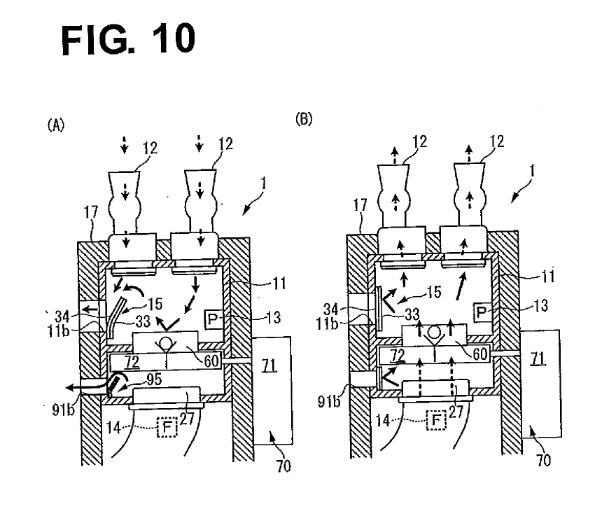

As shown in FIG. 10, as an application of the present embodiment, an exhaust hole 91b and an exhaust valve 95 are preferably further provided between the blower 10 and the backflow prevention mechanism (check valve 60). The exhaust valve 95 is arranged in the inspiratory pathway to block the exhaust hole 91b. As shown in FIG. 10(A), the exhaust valve 95 opens the exhaust hole 91b at the timing of expiration to release the air supplied from the blower 10. As in FIG. 10(B), the air hole 11b is closed at the timing of inspiration to pass all the air (inspiratory air) from the blower 10 to the prong 12 side.

Consequently, during the expiration of FIG. 10(A), an increase in the internal pressure of the inspiratory pathway on the upstream side can be reduced even if the blower 10 is maintained ON while the check valve 60 blocks the backflow of the exhaled air. If the exhaust valve 95 is closed with the blower 10 ON, the air can be quickly supplied to the prongs 12 at the time of the inspiration of FIG. 10(B). This can also reduce the amount of variations in the flowrate of the blower 10, whereby fluctuations of motor noise can also be suppressed. As in the present example, the exhaust hole 91b and the exhaust valve 95 are desirably arranged upstream (blower 10 side) of the downstream humidifier 70 to release the air before the humidification to the atmosphere. This can prevent waste of moisture in the downstream humidifier 70.

Since the exhaust valve 95 may be opened and closed at the same timing as with the expiratory valve 15, the expiration detection by the air pressure meter 13 can be used to control the exhaust valve 95 by a controller. The expiratory valve 15 and the exhaust valve 95 may be integrated to open and close the air hole 11b and the exhaust hole 91b simultaneously by a single valve. The exhaust hole 91b may always be left open without the provision of the exhaust valve 95, and the flowrate of the blower 10 may be increased as much as the leakage from the exhaust hole 91b. Although not shown in particular, the air hole 11b and the exhaust hole 91b may preferably be arranged close to each other. When the exhaled air is released from the air hole 11b, the exhaust resistance of the exhaust hole 91b is induced to decrease by the flow of the exhaled air. When the exhaled air is not released from the air hole 11b, the exhaust resistance of the exhaust hole 91b increases.

The foregoing embodiment has been described by using the blower 10 including an impeller as an example of the gas supply source. However, the present invention is not limited thereto. For example, a micropump or the like may be included. A micropump is a pump using a diaphragm fixed to a piezoelectric element, and can force-feed air by vibrations of the diaphragm.

In the foregoing embodiment, the air pressure meter is described as an example of the sensor for detecting the exhaled air. However, a flow sensor for detecting a flow of the exhaled air can be used. Other sensors can also be used.

In the foregoing embodiment, the expiratory valve 15 is described to be arranged on the air hole 11b. However, the present invention is not limited thereto, and the air hole 11b may always be left open. The flowrate of the blower 10 may be increased as much as the air supplied from the blower 10 leaks from the air hole 11b during expiration.

The present invention is not limited to the foregoing respective embodiments, and various modifications may be made without departing from the gist and technical idea thereof.

More specifically, in the foregoing respective embodiments, the positions, sizes (dimensions), shapes, materials, directions, and numbers of respective components may be changed as appropriate.

### REFERENCE SIGNS LIST

1 respiratory assistance apparatus
10 blower
11 chamber (pathway)
12 prong
13 air pressure meter
15 expiratory valve
60 check valve
70 downstream humidifier
80 upstream humidifier

1. A respiratory assistance apparatus comprising:
a gas supply source configured to supply a gas;
a connection part that is connected to a nose or mouth and configured to supply the gas thereto;
an inspiratory pathway configured to make the gas supply source communicate with the connection part and guide the gas;
a backflow prevention mechanism that is arranged on the inspiratory path and configured to allow the gas of the gas supply source to pass to the connection part side and to prevent exhaled air discharged from the nose or mouth via the connection part from flowing into a side of the gas supply source; and
an air hole that is formed in a pathway constituting member constituting the inspiratory pathway and configured to discharge the exhaled air, the air hole being formed closer to the connection part than the backflow prevention mechanism is.

2. The respiratory assistance apparatus according to claim 1, wherein the backflow prevention mechanism is a check valve that operates mechanically by using a pressure or flow of the exhaled air.

3. The respiratory assistance apparatus according to claim 1, wherein the backflow prevention mechanism is an actuated valve that operates by using an electrical signal obtained by detecting the exhaled air.

4. The respiratory assistance apparatus according to claim 1, further comprising an expiratory valve configured to open and close the air hole, wherein
the expiratory valve closes the air hole during inspiration and opens the air hole during expiration.

5. The respiratory assistance apparatus according to claim 4, comprising an expiratory sensor configured to detect the exhaled air, wherein
the expiratory valve opens and closes according to expiration detection of the expiratory sensor.

6. The respiratory assistance apparatus according to claim 5, wherein the expiratory sensor is arranged on the pathway constituting member, closer to the connection part than the backflow prevention mechanism is.

7. The respiratory assistance apparatus according to claim 5, wherein the expiratory sensor is an air pressure meter.

8. The respiratory assistance apparatus according to claim 1, wherein an exhaust hole configured to release the gas of the gas supply source is formed in the inspiratory pathway between the gas supply source and the backflow prevention mechanism.

9. The respiratory assistance apparatus according to claim 8, further comprising an exhaust valve configured to open and close the exhaust hole, wherein
the exhaust valve closes the exhaust hole during inspiration and opens the exhaust hole during expiration.

10. The respiratory assistance apparatus according to claim 1, wherein the inspiratory path between the gas supply source and the connection part has a length of 500 mm or less.

11. The respiratory assistance apparatus according to claim 1, wherein the inspiratory path between the check valve and the connection part has a length of 300 mm or less.

12. The respiratory assistance apparatus according to claim 1, wherein the gas supply source is a blower, and the blower is fixed to a human body.

13. The respiratory assistance apparatus according to claim 12, wherein the blower is fixed to a head.

14. The respiratory assistance apparatus according to claim 1, comprising a humidifying device that is configured to humidify the gas and is arranged on the pathway constituting member, closer to the gas supply source than the backflow prevention mechanism is.
